(19) United States
(12) Patent Application Publication
(10) Pub. No.: US 2003/0105526 A1
(43) Pub. Date: Jun. 5, 2003

(54) High tibial osteotomy (HTO) wedge

(75) Inventors: Richard M. Bryant, Clemmons, NC (US); Alina M. Waite, Clemmons, NC (US)

Correspondence Address: BAKER BOTTS L.L.P., 2001 ROSS AVENUE, SUITE 600, DALLAS, TX, 75201-2980, US

(73) Assignee: AMEI Technologies Inc.

(21) Appl. No.: 10/304,231

(22) Filed: Nov. 26, 2002

Related U.S. Application Data
(60) Provisional application No. 60/334,447, filed on Nov. 30, 2001.

(51) Int. Cl. A61F 2/28

(52) U.S. Cl. 623/16.11

(57) ABSTRACT

A device and a method for performing a high tibial osteotomy repair is disclosed. The osteotomy device may have the general configuration of a horseshoe-shaped wedge. The device is inserted into an osteotomy site after the tibia is realigned. The device generally conforms to the anatomical cross section of the tibia and thereby enhances weight bearing on the extremity in a postoperative recovery period. The device may also accommodate the stem of a knee replacement prosthesis. The method for correcting tibial deformities includes using a wedge-shaped body composed of various materials and having variable thickness and angulation to fill a gap in the tibia created by an osteotomy.

### RELATED APPLICATION

This application claims the benefit, under 35 U.S.C. §119(e), of previously filed provisional application High Tibial Osteotomy (HTO) Wedge, Serial No. 60/334,447, filed Nov. 30, 2001.

### TECHNICAL FIELD

The present invention is related generally to an orthopedic medical device and surgical procedure and specifically to a device and method for tibial realignment in which a wedge is inserted into an opening in the tibia.

### BACKGROUND OF THE INVENTION

Articulation of the tibia and the femur of a normal human knee joint is not perfectly straight, but is generally bent outward or away from the center plane of the body. This condition is known as valgus, with normal considered to be at approximately six degrees. The tibia may also be bent in an inward direction, a condition known as varus. Marked bending in either direction can alter the mechanical axis of the leg resulting in load patterns that a knee cannot properly accommodate. Over time a patient may develop severe pain and restriction of movement associated with a varus or valgus deformity. Various operative methods to correct these articulation abnormalities have been developed.

A high tibial osteotomy is an operation that is performed to correct an abnormal articulation of the tibia and femur. These deformities may either be pre-existing or can result from osteoarthritis, traumatic injury, or previous knee surgery.

One method of performing a tibial osteotomy, the medial tibial osteotomy, requires incising the medial cortex of a tibia down to medullary bone, leaving the lateral aspect of the tibia intact. The lower portion of the tibia is then repositioned to correct a deformity with the intact lateral cortex of the tibia acting as a hinge.

Angulating the lower tibia portion separately from the upper portion of the tibia, after the bone is incised, forms a gap at the incision point. A significant amount of bone growth is required to fill this space. The patient's own bone, harvested through a separate incision, is commonly employed to help close this gap along with a metal plate.

### SUMMARY OF THE INVENTION

In accordance with teachings of the present invention, an apparatus and method are provided to substantially reduce or eliminate disadvantages and problems associated with previous osteotomy devices and associated procedures. One aspect of the present invention includes a wedge-shaped insert formed from durable biocompatible material that can substitute for a patient's autologous bone and eliminate any need to obtain bone from another part of the patient's body.

Existing osteotomy wedges, which often do not conform with the anatomical configuration of a tibia, may have limited weight-bearing potential. Enhancing the weight-bearing potential of a healing tibia by using an anatomically-designed osteotomy device constructed of weight-bearing material is one embodiment of this invention.

While anterior osteotomy wedges have been previously known, they are generally not designed to be compatible with later insertion of a knee replacement prosthesis that must be anchored in a proximal tibia. Previous osteotomy surgery can complicate later insertion of a knee replacement prosthesis and may require removal or adjustment of an implanted wedge in order to accommodate the prosthesis.

In accordance with one aspect of the present invention, an osteotomy device is provided which conforms generally to the cross-sectional anatomy of a tibia and may be constructed of durable weight-bearing material. An osteotomy device formed in accordance with teachings of the present invention promotes early weight bearing by a healing bone after an osteotomy repair. In addition the osteotomy device may be designed to accommodate the stem of a knee replacement prosthesis through a central opening in the device. Alternatively, rather than being configured to accommodate a stem, one embodiment of the invention may consist of a biodegradable wedge. This type of wedge would degrade over time and encourage bone ingrowth such that no mechanical impediment would be present if a later knee replacement prosthesis is required.

An osteotomy device may be formed in accordance with teachings of the present invention from a wide variety of materials such as steel alloys, titanium, cobalt chrome, ceramics and composite materials. The osteotomy device may be formed with an open-cell configuration such as coral or sponge that allows ingrowth of bone and tissue into the device and stabilizes the bone earlier in the healing process. The size of the holes desired in a porous material might influence the selection of a material such as sponge or coral and would depend on the desired degree of bone ingrowth. Hydroxyapatite, or some other biocompatible material, might be incorporated into any or all of these materials in order to facilitate bone growth.

An osteotomy device may also be formed from a biodegradable, bioerodable or bioresorbable material. For example, an osteotomy device may be formed from material selected from a group consisting of polymers or copolymers of lactide, glycolide, caprolactone, polydioxanone, trimethlene carbonate, polyorthoesters and polyetheylene oxide or any combination thereof.

In accordance with another aspect of the present invention a method is provided for correcting tibial deformities that includes using internal fixation procedures and filling a gap in the bone created by an osteotomy. More specifically, this gap may be filled with a modified horseshoe-shaped wedge formed from weight-bearing material of variable thickness and angulation. The wedge may be employed on either the medial, lateral, anterior, or posterior aspect of the tibia.

An alternate method, that could further reduce time required for healing, would entail using a wedge in conjunction with an external fixator. In some cases, an external fixator may be the preferred method of treating a particular deformity. To be able to combine stabilizing aspects of an external fixator with stability and healing advantages of an osteotomy wedge may result in a shorter overall healing period and allow earlier removal of an external fixator.

A wedge may be formed in accordance with teachings of the present invention with various angles and dimensions as required for treatment of a patient. Also, the number of screws and the angle of screw insertion used to secure a wedge within an osteotomy site may be varied as required for treatment of a patient. For example, in one application of the invention angle of approximately ten degrees may be formed by the slope of the wedge and a flat surface on which the wedge is placed. In another application of the invention, the angle of screw insertion into the wedge may be approximately thirty five degrees. In a further application of the invention the angle formed by screw insertion may be approximately equal to twenty-five degrees. Twenty-five and thirty-five degree angles are formed with reference to the flat surface of the wedge.

### BRIEF DESCRIPTION OF THE DRAWINGS

A more complete and thorough understanding of the present embodiments and advantages thereof may be acquired by referring to the following description taken in conjunction with the accompanying drawings, in which like reference numbers indicate like features, and wherein:

FIG. 1A is a schematic drawing showing an isometric view of an osteotomy device having a substantially horseshoe-shaped body according to one embodiment of the invention;

FIG. 1B is a schematic drawing in elevation showing an edge view of the device of FIG. 1A;

FIG. 1C is a schematic drawing in elevation showing another edge view of superior and inferior screw openings located on the medial aspect of a horseshoe-shaped body;

FIG. 1D is a schematic drawing in section showing the device of FIG. 1A and the angulation of the superior screw opening;

FIG. 1E is a schematic drawing in section showing the device of FIG. 1A and angulation of the inferior screw opening;

FIG. 1F is a schematic drawing showing an isometric view of an osteotomy wedge incorporating teachings of the present invention without a central opening;

FIG. 2A is a schematic drawing showing a transverse incision into a medial upper tibia, according to an embodiment of the invention;

FIG. 2B is a schematic drawing showing a portion of the tibia below the transverse incision illustrated in FIG. 2A repositioned at a predetermined angle, to form an opening capable of receiving an osteotomy device, according to an embodiment of the invention;

FIG. 2C is a schematic drawing showing an exploded view of an osteotomy device operable to be inserted into the osteotomy opening illustrated in FIG. 2B according to an embodiment of the invention;

FIG. 3A is a schematic drawing in section of a knee replacement prosthesis wherein a tibial stem can pass through an opening in an osteotomy wedge found in accordance with teachings of the present invention and be anchored into the proximal tibia; and

FIG. 3B is a schematic drawing with portion broken away of an osteotomy wedge with a knee replacement prosthesis stem inserted through a central opening.

### DETAILED DESCRIPTION OF THE INVENTION

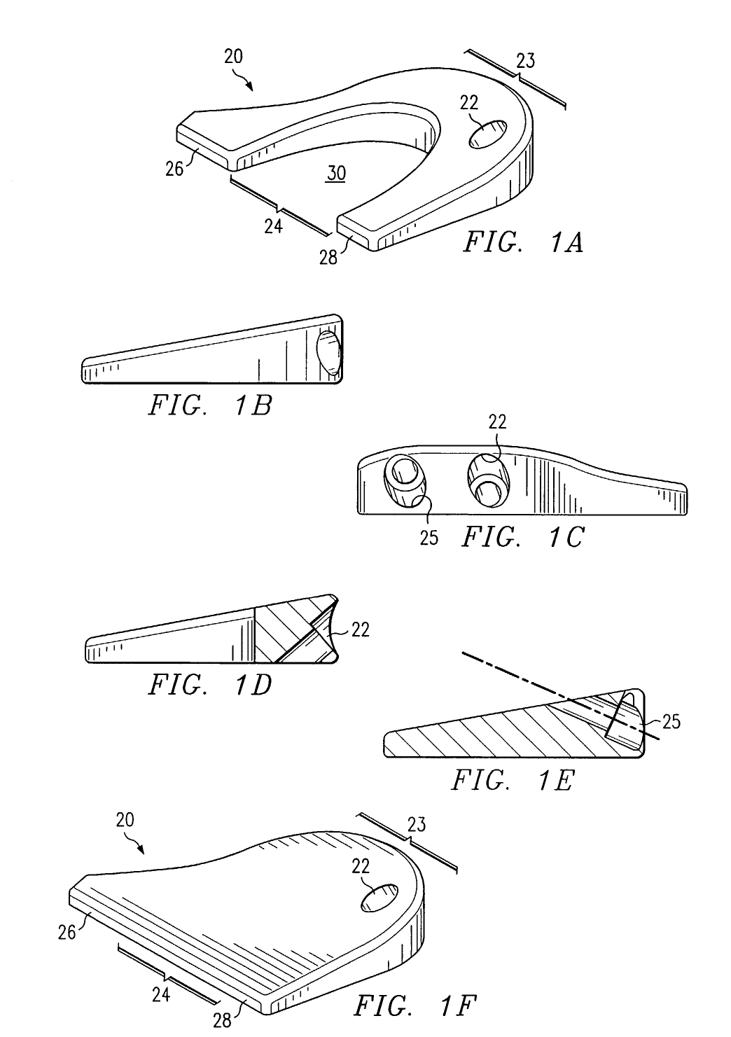

Preferred embodiments of the invention and its advantages are best understood by reference to FIGS. 1A through 3B, which illustrate various osteotomy devices and methods incorporating teachings of the present invention. FIG. 1A shows osteotomy device 20 which may be generally described as a wedge with a modified horseshoe configuration (asymmetrical U-shaped) designed to generally conform to an anatomical cross section of a tibia thereby providing mechanical support to a substantial portion of a tibial surface. In an alternative design a biodegradeable wedge, conforming to a cross section of a tibia, may be used without a central opening 30(See for example FIG. 1F).

The medial aspect 23 of device 20 is characterized by a curvilinear contour. Superior screw opening 22 may be located on the medial aspect of the device 20. Inferior screw opening 25 may also be located on the medial aspect of device 20. On the lateral aspect 24 of the wedge 20 there is a posteriorly-oriented appendage 26 that conforms to the shape of a tibial bone in this location. The opposite end of the lateral aspect of the wedge 28 is positioned anteriorly. Central opening 30 may be located on the lateral aspect 24 of the wedge that is designed to accommodate a stem of a knee replacement prosthesis.

Typical configurations of osteotomy device 20 are shown in FIGS. 1B-1E including various angles between the superior surface and the inferior surface of the wedge depending on its configuration. The thickness 32 of the substantially wedge-shaped body may vary from five millimeters to twenty millimeters depending on patient size and degree of deformity. Degree of angulation between the superior and inferior surfaces of the wedge may also be varied.

For some embodiments, an osteotomy device made in accordance with teachings of the present invention may be secured within an osteotomy site without the use of screws (not expressly shown). For example, a plurality of ridges or serrations (not expressly shown) may be formed on the superior surface and/or inferior surface of the device. Also, sutures and/or staples may be used to secure an osteotomy device within or around an osteotomy site. For still other applications, the material used to form a device in accordance with teachings of the present invention may not require the use of any mechanical fasteners (screws, sutures, staples, serrations or ridges) to secure the device within an osteotomy site.

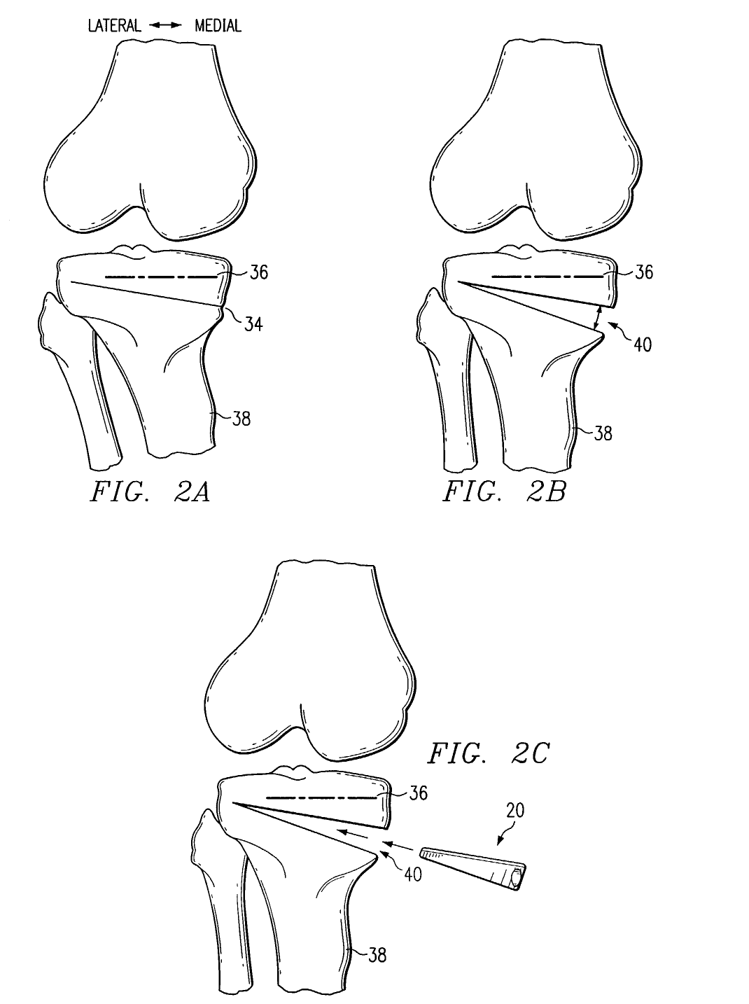

FIGS. 2A-2C illustrate one method of the present invention for realigning an abnormally angulated knee. In accordance with the method transverse incision 34 may be made into a medial aspect of a tibia as shown in FIG. 2A while leaving a lateral portion of the tibia intact.

In FIG. 2B the upper portion 36 and lower portions 38 of a tibia may be realigned at a predetermined angle to correct an angulation deformity. This creates space 40 between upper and lower portion 36 and 38 of a tibia. In FIG. 2C, the substantially wedge-shaped body of an osteotomy device 20 may be inserted into a space 40. Osteotomy wedge 20 serves the purpose of stabilizing two portions of a tibia as they heal into the desired position.

The principal surfaces of osteotomy wedge 20 substantially engage tibial surfaces thus promoting bone growth into the principal surfaces and minimizing instability in a healing osteotomy space. In one embodiment a wedge is composed of a weight-bearing material such as stainless steel that provides maximum support with maximum resistance to postoperative infection. In another embodiment of the invention a wedge is composed of an open-cell-configuration material such as sponge or coral which would maximally promote ingrowth of bone into the wedge and enhance healing and stability of the osteotomy site.

In yet another embodiment, a wedge may be formed from biodegradable, bioerodable or bioresorbable material, which, over time, will result in a wedge being reabsorbed and bone ingrowth occurring at an osteotomy site. For example, an osteotomy device may be formed from material selected from a group consisting of polymers or copolymers of lactide, glycolide, caprolactone, polydioxanone, trimethlene carbonate, polyorthoesters and polyetheylene oxide or any combination thereof. In this configuration, an osteotomy wedge would not require a central opening as shown in FIG. 1F. Advantages of this design include forming a wedge with optimum thickness resulting in increased strength and stability and reduced manufacturing costs.

Two screws may be used to fix an osteotomy wedge to a tibial bone in accordance with teachings of the present invention. FIG. 1C, an edge view of the osteotomy device of FIG. 1A, shows a superior screw hole 22 and inferior screw hole 25. Screws (not expressly shown) may be inserted at an angle depending on the degree of correction required and desired angulation of a wedge into an osteotomy opening as shown in FIG. 1D (superior screw opening 22) and FIG. 1E (inferior screw opening 25). In an alternate embodiment, only one screw may be required to secure a wedge to an osteotomy site.

Additional uses of an osteotomy wedge include as an adjunct to healing when an external fixator (not expressly shown) is required to correct alignment of limb and/or secure two healing ends of bone. Employing a wedge in conjunction with an external fixator may provide increased stabilization and may shorten a bone's healing period. An external fixator may possibly then be removed sooner.

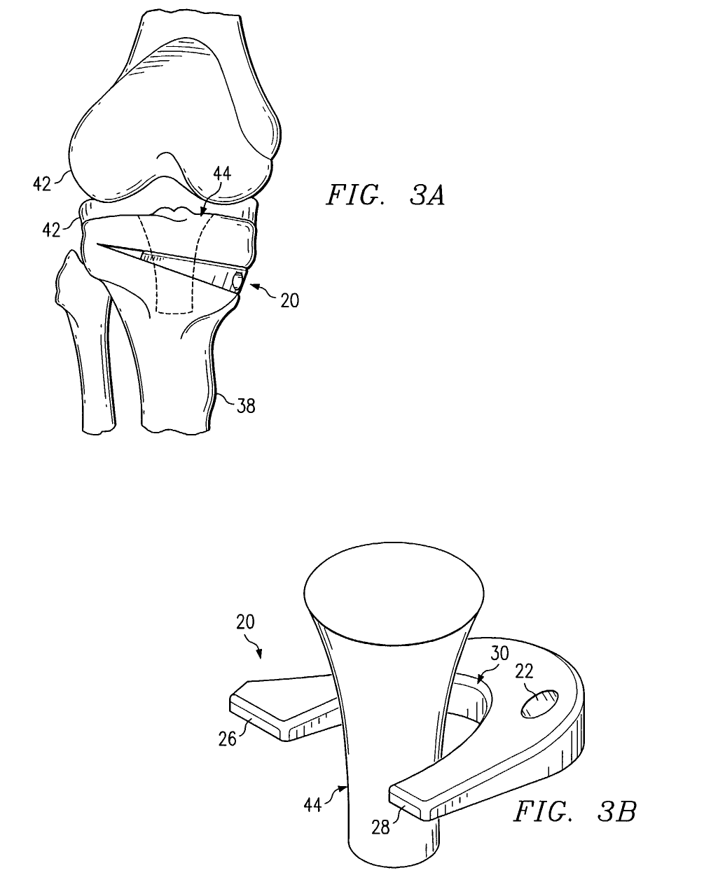

Osteotomy wedge 20 with central opening 30 may allow insertion of knee replacement prosthesis 42 as shown in FIGS. 3A and 3B. FIG. 3A shows how stem 40 of knee replacement prosthesis 42 may be inserted through central opening 30 of osteotomy wedge 20 and anchored into tibial bone 38. FIG. 3B shows how stem 40 of knee replacement prosthesis 42 passes through central opening 30 of osteotomy wedge 20. This embodiment has the advantage of allowing insertion of knee replacement prosthesis 42 after osteotomy wedge 20 with central opening 30 has been placed in a patient without first removing osteotomy wedge 20.

Although the present invention has been described with respect to a specific preferred embodiment thereof, various changes and modifications may be suggested to one skilled in the art and it is intended that the present invention encompass such changes and modifications fall within the scope of the appended claims.

What is claimed is:

1. An osteotomy device comprising;
a modified horseshoe-shaped body of variable thickness;
the body variably angulated from front to back;
the thickness and angulation of the body selected to allow insertion into a generally wedge-shaped osteotomy site; and
the modified horseshoe-shape operable to accommodate a stem of a knee replacement prosthesis through a central opening.

2. The osteotomy device of claim 1 further comprising material selected from the group consisting of stainless steel, titanium, cobalt chrome, ceramics and composites or an open-cell configuration material such as coral or sponge or any combination thereof.

3. The osteotomy device of claim 2 further comprising material selected from the group consisting of hydroxyapatite or another biocompatible material.

4. The osteotomy device of claim 1 further comprising material selected from the group consisting of a biodegradable, bioerodable or bioresorbable material.

5. The osteotomy device of claim 1 further comprising a material selected from the group consisting of polymers or copolymers of lactide, glycolide, caprolactone, polydioxanone, trimethylene carbonate, polyorthoesters and polyethylene oxide or any combination thereof.

6. The osteotomy device of claim 1 further comprising a weight-bearing material.

7. The osteotomy device of claim 1 further comprising a body having a variable thickness ranging from five millimeters to twenty millimeters.

8. The osteotomy device of claim 1 further comprising a body of variable angulation.

9. An osteotomy device comprising;
a generally asymmetrical U-shaped body of variable thickness;
the body having variable angulation from front to back; and
the body operable to be inserted horizontally into a wedge-shaped osteotomy site.

10. The osteotomy device of claim 9 further comprising stainless steel, titanium, cobalt chrome, ceramics and composites or an open-cell configuration material such as coral or sponge or any combination thereof.

11. The osteotomy device of claim 9 further comprising hydroxyapatite or another biocompatible material.

12. The osteotomy device of claim 9 further comprising a biodegradable, bioerodable or bioresorbable material.

13. The osteotomy device of claim 9 wherein said biodegradable, bioresorbable or bioerodable material comprises material selected from the group consisting of polymers or copolymers of lactide, glycolide, caprolactone, polydioxanone, trimethylene carbonate, polyorthoesters and polyethylene oxide or any combination thereof.

14. The osteotomy device of claim 9 further comprising a weight-bearing material.

15. The osteotomy device of claim 9 further comprising a body of variable thickness ranging from five millimeters to twenty millimeters.

16. A method for correcting tibial deformities comprising:
forming an osteotomy in a bone;
filing a gap in the bone created by an osteotomy with a modified horseshoe-shaped wedge; and
attaching the wedge to at least one portion of the bone.

17. The method of claim 16 comprising using one screw to attach the wedge to a tibia.

18. The method of claim 16 comprising using two screws to attach the wedge to a tibia.

19. The method of claim 16 comprising inserting a bone pin through an opening in the wedge.

20. The method of claim 16 comprising growing bone through porous spaces in the wedge.
